(19) United States
(12) Patent Application Publication
(10) Pub. No.: US 2017/0182272 A1
(43) Pub. Date: Jun. 29, 2017

(54) SMART ORONASAL MASK FOR POSITIVE-AIRWAY-PRESSURE DEVICE

(71) Applicant: SLS MEDICAL TECHNOLOGY CORP. LTD., TAICHUNG CITY (TW)

(72) Inventor: CHING-LUNG SUNG, TAICHUNG CITY (TW)

(21) Appl. No.: 15/040,316

(22) Filed: Feb. 10, 2016

(30) Foreign Application Priority Data
Dec. 28, 2015 (TW) 104220926

(51) Int. Cl. A61M 16/06; A61M 16/00

(52) U.S. Cl. CPC A61M 16/06; A61M 16/0003; A61M 2205/3576; A61M 2205/3368

(57) ABSTRACT

A smart oronasal mask for a positive-airway-pressure device includes a mask body and a temperature sensor. The mask body has an air inlet and an air outlet. The temperature sensor is deposited in the mask body and located between the air inlet and the air outlet for sensing a temperature of a mixture formed by gas entering the mask body through the air inlet of the mask body and gas entering the mask body through the air outlet of the mask body. Then the positive airway pressure device adjusts air feed according to the temperature sensed by the temperature sensor in a real-time manner, so as to help keep a patient's respiratory tract clear.

### BACKGROUND OF THE INVENTION

1. Technical Field

The present invention relates to positive airway pressure devices, and more particularly to a smart oronasal mask for a positive-airway-pressure device.

2. Description of Related Art

Obstructive sleep apnea, also known as OSA is a sleep disorder that involves repeated cessation in airflow in the presence of breathing effort. Such apnea is caused by excess throat muscle texture or abnormal oral & maxillo-facial structure that makes the muscles too weak to keep the respiratory tract clear. In both case, the respiratory tract collapses during sleep and hinders air from entering the lungs adequately. As a result, the blood oxygen saturation decreases dramatically, so the patient rouses suddenly from sleep by his/her brain. A patient with OSA not only suffers from poor sleep quality, but also is bothered by daytime distraction, hypersomnia and attention deficit. In the worse cases, the patient may have cardiovascular diseases due to insufficient blood oxygen.

Currently, one effective treat to OSA is the use of positive airway pressure devices that keep a patient's respiratory tract clear during sleep. In operation, an oronasal mask is put on a patient's oronasal portion, and then the positive airway pressure device operates and sends air into the patient's respiratory tract through the oronasal mask, so as to prevent the respiratory tract from collapsing. However, for the patients who still have unstable breath or even apnea when using a positive airway pressure device, the existing positive airway pressure devices can only feed oxygen as preprogrammed, but is not capable of detecting patients' abnormal breath and adjusting their air feed accordingly. For this reason, the existing positive airway pressure devices have only limited efficacy.

### BRIEF SUMMARY OF THE INVENTION

The primary objective of the present invention is to provide a smart oronasal mask for a positive-airway-pressure device, wherein the smart oronasal mask senses a temperature difference between the gas inhaled by a patient and the gas breathed out by the patient, and make the positive airway pressure device adjust air feed according to the sensing result.

For achieving the foregoing objective, the smart oronasal mask of the present invention comprises a mask body and a temperature sensor. The mask body has an air inlet and an air outlet. The temperature sensor is deposited in the mask body and located between the air inlet and the air outlet for sensing a temperature of a mixture formed by gas entering the mask body through the air inlet of the mask body and gas entering the mask body through the air outlet of the mask body, and transmitting the sensing result to the positive airway pressure device for determination. The positive airway pressure device then adjusts air feed according to the sensing result in a real-time manner, so as to help keep a patient's respiratory tract clear during the patient's sleep, thereby providing good therapeutic efficacy.

The following preferred embodiments when read with the accompanying drawings are made to clearly exhibit the above-mentioned and other technical contents, features and effects of the smart oronasal mask. Through the exposition by means of the specific embodiments, people would further understand the technical means and effects the present invention adopts to achieve the above-indicated objectives. However, the accompanying drawings are intended for reference and illustration, but not to limit the present invention.

### BRIEF DESCRIPTION OF THE SEVERAL VIEWS OF THE DRAWINGS

FIG. 1 is a perspective view of a smart oronasal mask of the present invention.

FIG. 2 is a block diagram of the smart oronasal mask of the present invention.

FIG. 3 is another block diagram of the smart oronasal mask of the present invention.

FIG. 4 graphically shows temperature variation over time in connection to use of the smart oronasal mask of the present invention.

### DETAILED DESCRIPTION OF THE INVENTION

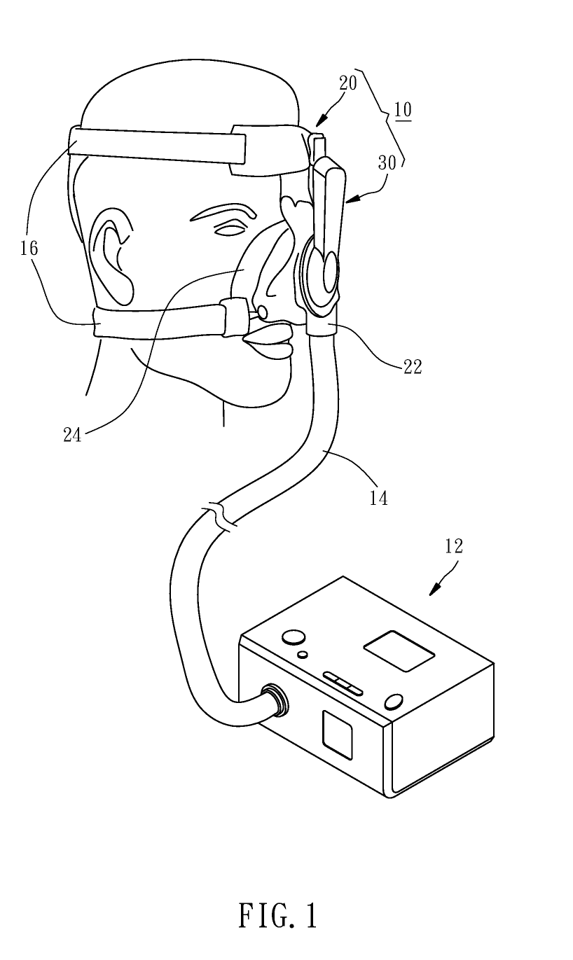

Referring to FIG. 1, the disclosed smart oronasal mask 10 comprises a mask body 20 and a temperature sensor 30.

The mask body 20 has an air inlet 22 and an air outlet 24. The air inlet 22 is connected to a positive airway pressure device 12 through an air hose 14. The mask body 20 is configured to be put on a patient's head with two head straps 16. When put on by a patient, the air outlet 24 of the mask body 20 well covers the patient's nose. Thereby, the air fed by the positive airway pressure device 12 goes through the air hose 14 to the air inlet 22 of the mask body 20, and enters the mask body 20 via the air inlet 22 of the mask body 20 before inhaled by the patient through the air outlet 24 of the mask body 20. On the other hand, the gas breathed out by the patient enters the mask body 20 through the air outlet 24 of the mask body 20.

The temperature sensor 30 is installed in the mask body 20 and is located between the air inlet 22 of the mask body 20 and the air outlet 24 of the mask body 20, The temperature sensor 30 is such positioned that it senses a temperature of a mixture formed by the gas entering the mask body 20 through the air inlet 22 of the mask body 20 and the gas entering the mask body 20 through the air outlet 24 of the mask body 20, and transmits the sensing result to the positive airway pressure device 12 for determination. It is to be noted that the gas entering the mask body 20 through the air inlet 22 of the mask body 20 refers to the gas output by the positive airway pressure device 12, and the gas entering the mask body 20 through the air outlet 24 of the mask body 20 refers to the gas breathed out by the patient. In terms of gas temperature, the gas output by the positive airway pressure device 12 has a temperature close to be room temperature, while the gas breathed out by the patient has a temperature slightly lower than the human body temperature since it just past the patient's respiratory tract, which is approximately 32° C.˜34° C. on average. Thus, the temperature of the mixture of the two streams of gas serves as an indication for whether the patient is breathing out or breathing in. For example, when the temperature is close to and not exceeds 32° C.˜34° C., the patient is breathing out. In a different instance, when the temperature is close to and not lower than the room temperature, the patient is breathing.

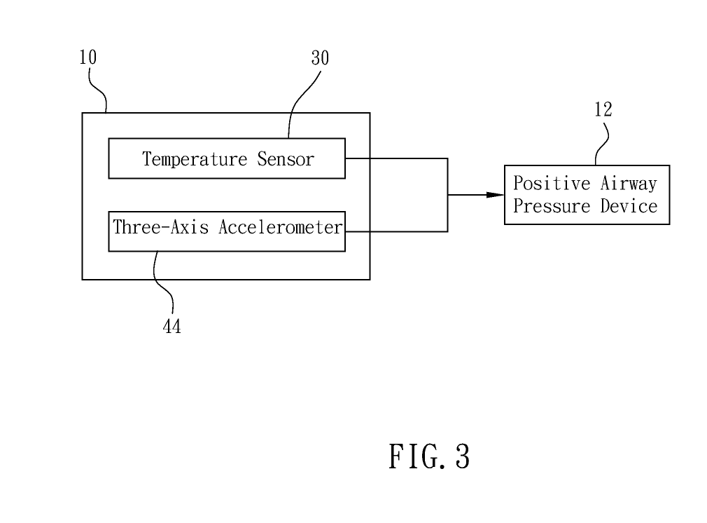

FIG. 3 is a wave graph of a patient's breath measured by the temperature sensor 30 and contains multiple peaks P1 and multiple valleys P2. Each of the peaks P1 represents a relative high temperature that occurs when the patient is breathing out, and each of the valleys P2 represents a relative low temperature that occurs when the patient is breathing in. For a person who breathes normally, the peaks P1 and the valleys P2 continuously alternate in a certain temperature range. However, for a person with apnea, discontinuity P3 appears in the waveform at the point where apnea happens.

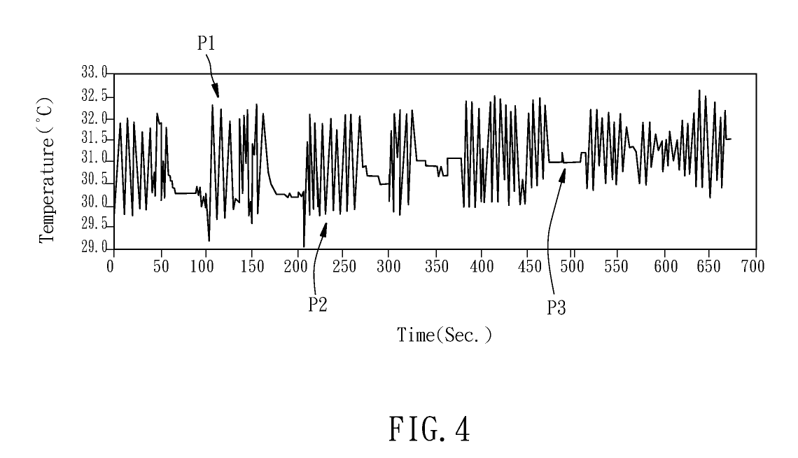

In addition to showing the patient's breathing state, the data from FIG. 4 also informs of various abnormalities as they appear. For instance, where the value stays about the height of the peaks P1, the high temperature indicates that the gas in the mask body 20 mainly comes from the patient's exhalation. In other words, there may be not enough gas entering the mask body from the positive airway pressure device 12, and this may because of problems with the air hose 14 or with the device itself. On the other hand, where the value stays about the height of the valleys P2, the low temperature indicates that the gas in the mask body 20 mainly comes from the positive airway pressure device 12, and this may indicate misalignment or leakage of the mask body 20.

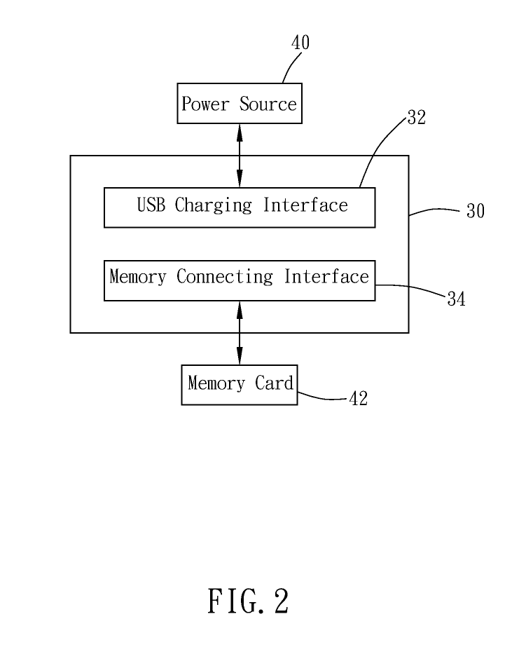

To sum up, the disclosed smart oronasal mask 10 measures the patient's breathing state at a point near the patient's oronasal portion, and detects the patient's breathing abnormalities using the temperature variation between air feed and the patient's exhalation. As compared to the prior art, the present invention provides more accurate sensing results that facilitate real-time adjustment of the air feed from the positive airway pressure device 12, so as to keep the patient's respiratory tract clear during sleep and thereby obtain good therapeutic efficacy. Furthermore, as shown in FIG. 2, the temperature sensor 30 has a USB charging interface 32, which is configured to connect an external power source 40 for charging, so as to simplify its wiring structure. The temperature sensor 30 further has a memory connecting interface 34 for receiving and connecting a memory card 42 that store all the data collected during the use of the mask for later analysis. Referring to FIG. 3, the disclosed smart oronasal mask 10 has a built-in three-axis accelerometer 44 for sensing an angular difference between the oronasal mask 10 and the ground, and transmitting the sensing result to the positive airway pressure device 12, so that the positive airway pressure device 12 can use the data together with the temperature recording for further comparison and analysis.

What is claimed is:

1. A smart oronasal mask for a positive-airway-pressure device, the smart oronasal mask comprising:
a mask body, having an air inlet and an air outlet; and
a temperature sensor, deposited in the mask body and located between the air inlet and the air outlet for sensing a temperature of a mixture formed by gas entering the mask body through the air inlet of the mask body and gas entering the mask body through the air outlet of the mask body.

2. The smart oronasal mask of claim 1, wherein the temperature sensor has a USB charging interface.

3. The smart oronasal mask of claim 1, wherein the temperature sensor has a memory connecting interface.

4. The smart oronasal mask of claim 1, further comprising a three-axis accelerometer that is deposited in the mask body for sensing an angular difference between the smart oronasal mask and the ground.
